(19) United States
(12) Patent Application Publication
(10) Pub. No.: US 2023/0041965 A1
(43) Pub. Date: Feb. 9, 2023

(54) CULTURE DEVICE CONTAINING OXYGEN SENSITIVE LUMINOPHORE AND METHODS OF USING

(71) Applicant: 3M INNOVATIVE PROPERTIES COMPANY, St. Paul, MN (US)

(72) Inventors: Neil Percy, St. Paul, MN (US); Wensheng Xia, Woodbury, MN (US); Adam J. Stanenas, Cottage Grove, MN (US)

(21) Appl. No.: 17/758,719

(22) PCT Filed: Feb. 8, 2021
(86) PCT No.: PCT/IB2021/051010

§ 371 Date: Jul. 13, 2022

Related U.S. Application Data
(60) Provisional application No. 62/976,701, filed on Feb. 14, 2020.
(60) Provisional application No. 63/048,717, filed on Jul. 7, 2020.

(51) Int. Cl. C12Q 1/06; C12M 1/12; B01L 3/00

(52) U.S. Cl. CPC C12Q 1/06; C12M 25/06; B01L 3/508; B01L 2200/16; B01L 2200/0689

(57) ABSTRACT

A culture device comprising an oxygen sensitive luminophore, typically an oxygen sensitive phosphor, such as a porphyrin, and methods of use for culturing and enumerating microorganisms

### BACKGROUND

The article “Non-invasive transdermal two-dimensional mapping of cutaneous oxygenation with a rapid drying liquid bandage” (Li et al.) discloses that oxygen-dependent phosphorescence emission of a bandage has been used to quantify and map both pO₂ and oxygen consumption and oxygen consumption of the underlying tissue.

The article “The triplet state in Pt-acetylide oligomers, polymers and copolymers” (Silverman et al.) discloses that platinum acetylide oligomers and polymers are pi-conjugated materials that display luminescence from the triplet exciton.

The article “Conjugated-Polymer-Amplified Sensing, Imaging, and Therapy (Wu et al.) discloses that conjugated polymers are a key platform for amplifying detection signatures that betray the presence of biomarkers.

The article “irreversible sensing of oxygen ingress” (Wilhelm et al.) discloses two different absorption-based irreversible but regenerable optical probes for oxygen.

U.S. Pat. No. 3,338,794 discloses inexpensive, disposable devices for the culturing or anaerobic microorganisms that do not require the use of costly and time-consuming techniques for the removal of oxygen prior to the incubation period.

US20180312895 discloses a device for enumerating colonies of microorganisms. Disposed within the growth compartment of the device are a cold water-soluble gelling agent, a dry oxygen-scavenging reagent, a dry buffer system, and an effective amount of a dry carbon dioxide-generating reagent.

### DETAILED DESCRIPTION

Throughout this disclosure, singular forms such as “a,” “an,” and “the” are often used for convenience; however, the singular forms are meant to include the plural unless the singular alone is explicitly specified or is clearly indicated by the context. When the singular alone is called for, the term “one and only one” is typically used.

Some terms in this disclosure are defined below. Other terms will be familiar to the person of skill in the art and should be afforded the meaning that a person of ordinary skill in the art would have ascribed to them.

Terms indicating a high frequency, such as (but not limited to) “common,” “typical,” and “usual,” as well as “commonly,” “typically,” and “usually” are used herein to refer to features that are often employed in the invention and, unless specifically used with reference to the prior art, are not intended to mean that the features are present in the prior art, much less that those features are common, usual, or typical in the prior art.

The term “oxygen sensitive dye” refers to a chemical entity that changes the wavelength or intensity of light that it absorbs or emits in the presence of oxygen. A compound that neither absorbs nor emits light in the absence of oxygen but does absorb or emit light in the presence of oxygen is one type of “oxygen sensitive dye.” Oxygen sensitive luminophores (as defined herein), as well as oxygen sensitive phosphors (as defined herein) and colorimetric oxygen dyes (as defined herein) are examples of oxygen sensitive dyes.

The term “colorimetric oxygen dye” refers to a chemical entity that changes the wavelength at which it absorbs light (such as the wavelength of maximum absorption, or λₘₐₓ), particularly ultraviolet or visible light, in the presence of oxygen (as opposed to in the absence of oxygen). The change need not be reversible. Particular colorimetric oxygen dyes do not absorb sufficient light to be visible to a human eye in the absence of oxygen, but upon exposure do absorb sufficient light to be visible to a human eye; other particular colorimetric oxygen dyes have a first λₘₐₓ in the absence of oxygen and a second, different λₘₐₓ after exposure to oxygen. In either case, the change may be reversible in that the colorimetric oxygen dye may revert to its pre-oxygen exposure state if oxygen is removed, or irreversible.

The term “luminophore” refers to a chemical entity that exhibits luminescence.

The term “oxygen sensitive luminophore” refers to a luminophore having luminescence that is quenched in the presence of oxygen.

The term “phosphor” refers to a luminophore that exhibits phosphorescence. A phosphor may also exhibit fluorescence, but this is not required.

The term “oxygen sensitive phosphor” refers to a phosphor having phosphorescence that is quenched in the presence of oxygen. If the phosphor exhibits fluorescence, the fluorescence may also be quenched by the presence of oxygen, but this is not required.

An “oxygen scavenging system” refers to a chemical, biological, or mechanical system, which may be an enzymatic or other chemical system, that is designed to consume oxygen, typically substantially all of the oxygen, within a growth compartment of a culture device. However, an oxygen scavenging system does not include microorganisms that are being cultured on a culture device, such as in a growth compartment of a culture device.

The verb “quench” and its conjugates mean to cause a decrease in luminescence intensity; when used in relationship with a phosphor or phosphorescence it means more specifically to cause a decrease in phosphorescence intensity. Thus, if a phosphor is quenched by oxygen, then the intensity of phosphoresce of the phosphor decreases with increasing partial pressure of oxygen.

This disclosure recognizes that, in the technology of culturing and detecting microorganisms, problems exist in that it is often necessary to stain or otherwise impart color to the microorganisms being cultured. Even when staining is not required, it may be necessary to rely on detecting an intrinsic color of the microorganism. In either case, detection relies on a light source that is external to the culture device, such as a lamp or other source of illumination in the detector. This increases the cost of detectors, which have to be built not only to have special lamps for illuminating culture devices, but also have to be configurable to repeatably provide identical illumination conditions in order to provide consistent results. The problem is even more difficult when microorganisms are to be enumerated, because the illumination conditions must be highly repeatable in order to ensure that the enumeration is correct.

A related problem is how to use oxygen-sensitive dyes to detect, and more particularly to enumerate, cultured microorganisms.

A related problem is how to use emitted light to detect, and more particularly to enumerate, cultured microorganisms.

This disclosure also recognizes a problem in the field of air sensitive phosphors, and more specifically oxygen sensitive phosphors. Thus, another problem is how to use an oxygen-sensitive luminophore, and more particularly an oxygen-sensitive phosphor, to detect the presence of cultured microorganisms. A related problem is how to use porphyrin containing materials to detect, and more particularly to enumerate, cultured microorganisms.

This disclosure also recognizes a problem in the field of colorimetric oxygen dyes. Thus, another problem is how to use a colorimetric oxygen dye to detect the presence of cultured microorganisms.

These and related problems are addressed by the use of a culture device as described herein. The culture device has a growth compartment that is surrounded by one or more oxygen impermeable barriers. At least one of the oxygen impermeable barriers is configurable between an open configuration and a closed configuration. In the open configuration, the growth compartment is exposed to an environment outside of the growth compartment. In the closed configuration, the growth compartment is sealed from exchanging oxygen with the environment outside of the growth compartment.

The culture device also includes a culture medium capable of supporting replication of at least one microorganisms disposed within the growth compartment. Also, an oxygen-sensitive dye, particularly a colorimetric oxygen dye or an oxygen-sensitive luminophore, and more particularly an oxygen-sensitive luminophore, is disposed within the growth compartment.

Various embodiments of the culture device as well as methods described herein can be used to address the foregoing problems and other problems.

In any of the culture devices described herein, the one or more oxygen impermeable barriers can be those that are employed in the 3M™ Petrifilm™ Lactic Acid Bacteria Count Plates (available from 3M Company, St. Paul, Minn., USA). The oxygen impermeable barriers may include such materials as polyethylene, for example low density polyethylene, linear low density polyethylene, and the like, foil, such as aluminum foil, and other oxygen impermeable materials known in the art; one material or a combination of materials can be used to create the oxygen impermeable barrier.

Relating to any of the aforementioned culture devices, at least one of the oxygen impermeable barriers favorably comprises a cover slip. In any culture device where a cover slip is present, the open configuration can be a configuration wherein the coverslip is on the growth compartment and the closed configuration can be a configuration wherein the coverslip is at least partially detached from the growth compartment.

In any of the aforementioned culture devices, a port can be present in at least one of the one or more oxygen impermeable barriers such that the port can be converted between an open and closed configuration. For example, the growth compartment can be inoculated when the port is in an open configuration, after which the port can be closed.

With regard to any of the foregoing culture devices, the culture medium can be any type of culture medium and may be varied depending on the type of microorganism to be cultured, the detection method to be used, or other practical considerations. For example, in any of the foregoing embodiments of the culture device, the culture medium can be a thin-film culture medium, and more particularly a cold-water gelling thin film culture medium. Culture media of this type are commercially available, such as under those sold under the PETRIFILM™ brand by 3M Company St. Paul, Minn. USA. As an alternative, agar can be used as the medium in any of the aforementioned culture devices.

With regard to any of the culture devices described herein, any suitable oxygen-sensitive dye can be used. Examples of oxygen sensitive dyes include colorimetric oxygen dyes and oxygen-sensitive luminophores.

Oxygen-sensitive luminophores are particular oxygen-sensitive dyes that can be employed. With regard to any of the culture devices described herein, the oxygen-sensitive luminophore can be any luminophore that is quenched by oxygen. Favorably, in any culture device the oxygen sensitive luminophore is an oxygen sensitive phosphor. Regarding any aforementioned culture device, the oxygen sensitive phosphor can favorably comprise at least one of a porphyrin, or a pi-conjugated molecule, or a pi-conjugated polymer. With regard to any of the culture devices described herein, the oxygen sensitive phosphor can comprise a dendrimer. With regard to any of the culture devices described herein, the oxygen sensitive phosphor can comprise a porphyrin. With regard to any of the culture devices described herein, the oxygen sensitive phosphor can comprise a pi-conjugated molecule. In any of the disclosed culture devices where a pi-conjugated molecule is employed, the pi conjugated molecule is favorably comprises a pi-conjugated ligand for a transition metal or a lanthanide. Examples of these include cyclometallated complexes of iridium (III) or platinum (II), and particularly pyridine, such as 2-substituted pyridine, particularly aryl or cycloaryl pyridine, and even more particularly phenyl pyridine complexes of iridium (III) or platinum (II). Other examples include pyridine-based, and more particularly polypyridyl complexes of ruthenium (II), osmium (II), or rhenium (II). The pi-conjugated ligand, in any of the culture devices in which it is employed, can be a bipyridine. By “a bipyridine” it is meant that the bipyridine moiety is present in the molecule, but other moieties may or may not additionally be present, and in the case when other moieties are present they are directly or indirectly bound to the bipyridine moiety. The pi-conjugated ligand, in any of the culture devices in which it is employed, can be an acetylide. In any of the culture devices wherein an acetylide is employed, the acetylide can be a phenylene ethynylene or a poly phenylene ethynylene. By “a phenylene ethynylene or a poly phenylene ethynylene” it is meant that the phenylene ethynylene or poly phenylene ethynylene moiety is present in the molecule, but other moieties may or may not additionally be present, and in the case when other moieties are present they are directly or indirectly bound to the phenylene ethynylene or poly phenylene ethynylene moiety. The pi-conjugated ligand, in any of the culture devices in which it is employed, can be a porphyrin. The pi-conjugated ligand, in any of the culture devices in which it is employed, can be a dendrimer. Favorably, the pi-conjugated ligand, in any of the culture devices in which it is employed, can be a porphyrin containing dendrimer.

With regard to any of the culture devices mentioned herein, a metal can be conjugated to the oxygen sensitive luminophore, which can be any of the oxygen sensitive luminophores mentioned herein, and more particularly to a pi-conjugated molecule. With regard to any case wherein a metal is conjugated to a pi-conjugated molecule, the metal is favorably a transition metal or a lanthanide, though other metals, such as actinides, may also be used. Transition metals are most commonly used when a metal is conjugated to the pi-conjugated molecule. In any culture device where a metal is conjugated to any luminophore, the conjugation can be by any type of chemical interaction, such as ligation, covalent bonding, ionic bonding, van der Waals interactions, and the like.

With regard to any of the heretofore mentioned culture devices, the transition metal that is conjugated to the pi-conjugated molecule, when employed, is favorably selected from palladium, platinum, rhenium, or ruthenium. However, it should be understood that other transition metals may also be used. In any culture device wherein a lanthanide is used, the lanthanide is most commonly iridium. It is to be understood that in all cases wherein the oxygen sensitive phosphor comprises a metal, including cases where the metal is a transition metal, lanthanide, or other, palladium, platinum, rhenium, or ruthenium, or iridium, the metal may be in any oxidation state that provides an oxygen sensitive phosphor, and is not necessarily in the zero oxidation state.

When an acetylide is employed in any culture device described herein as a pi-conjugated ligand, the acetylide is favorably conjugated to a platinum metal.

Particularly in any herein-described culture device, a porphyrin containing oxygen sensitive phosphor can be used. In any of the culture devices employing a porphyrin-containing oxygen sensitive phosphor, the porphyrin can be conjugated to a metal, such as the any of the metals discussed above. The porphyrin containing oxygen sensitive phosphor in any culture device disclosed herein can be a porphyrin dendrimer. Most particularly the porphyrin dendrimer, in any culture device described herein, can be coordinated to a metal, the metal particularly being a transition metal or lanthanide, and most particularly being platinum or palladium. Porphyrin containing dendrimers have been disclosed. A particular porphyrin containing dendrimer that can be used in any of the aforementioned culture devices is Pd-meso-tetra-(4-carboxypenyl)porphyrin dendrimer, which is known in the art and can be made by art recognized methods. Other porphyrins and porphyrin containing dendrimers, as well as the other types of oxygen sensitive phosphors described herein for use with culture devices, can also be made according to art recognized methods.

Other examples of oxygen sensitive phosphor that can be used include, without limitation, phosphorescent Al(III)-ferron complexes, phosphorescent boron complexes, complexes of rare earth elements or salts thereof, Cu(I), Au(I), and the like.

Oxygen-sensitive dyes that are not luminophores include, without limitation, leuco-form indigo dye, leuco-form thioindigo dye, one or more complexes of bis(histadino) cobolt, meso-tetra(α-α-α-α-o-pivalminophenyl) porphyrinatocobolt, and fullerenes such as Buckminster fullerenes. Still others include polycyclic aromatics, such as 1-pyrenedecanoic acid and decacyclene.

In any of the culture devices described herein, any of the aforementioned oxygen sensitive dyes, and particularly any of the aforementioned oxygen-sensitive luminophores, can be disposed within the culture medium.

In any of the aforementioned culture devices, an adhesive may be present within the growth compartment and, in any case where an adhesive is present, any of the oxygen sensitive dyes or luminophores described herein can be disposed within or on the adhesive.

Any of the aforementioned culture devices, which may contain any of the aforementioned oxygen sensitive dyes, and particularly oxygen-sensitive luminophores, will favorably not contain an oxygen scavenging system within the growth compartment. As noted above, microorganisms to be cultured, such as microorganism that may be used to inoculate any culture device describe herein, are not considered an oxygen scavenging system in this disclosure. A volume of oxygen is favorably present within the atmosphere of the growth compartment in any of the culture devices described herein. Particularly, when the growth compartment is disposed in the closed configuration, the atmosphere within the growth compartment cannot communicate with the atmosphere outside the growth compartment. As a consequence, any oxygen within the growth department that is depleted cannot be restored by way of diffusion of oxygen from the exterior of the growth compartment to the interior of the growth compartment.

In use, any of the aforementioned culture devices, which may contain any of the oxygen sensitive luminophores described herein, can be provided in an open configuration and the growth compartment inoculated with a sample containing one or more microorganisms. In any method of use, the sample can be a liquid sample, particularly an aqueous liquid sample, that can be added to the growth compartment. Alternatively, in any method of use, the sample can be a swabbed sample, such as one located on an absorbent swab, that can inoculate the growth compartment by contacting the swab with the medium within the growth compartment.

With regard to any of the methods described herein, which can be employed with any of the herein described culture devices, the microorganism can be any microorganism that consumes oxygen. Typically this means that the microorganism will be an aerobe or a facultative anaerobe. However, it may also be possible to culture microaerophiles using the methods described herein.

After inoculation, the culture device can be converted to the closed configuration. In the closed configuration, the growth compartment initially has an oxygen content, which can be referred to or measured, for example, as the oxygen partial pressure, that is not dissimilar from that of the environment external to the growth compartment. This is so because the culture device was configured in the open configuration during the inoculation step.

The culture device is then incubated for a sufficient time and at a sufficient temperature such that the oxygen-sensitive dye, which can be any of the aforementioned oxygen sensitive dyes and particularly any of the aforementioned oxygen-sensitive luminophores, undergoes a change in absorption or emission, which in the case of an oxygen-sensitive luminophore is typically luminescence of the oxygen-sensitive luminophore. The time and temperature will vary depending on the particular microorganism that is being cultured. Typical times are from one hour to seven days, and typical temperatures are from 20 C to 60 C. When the oxygen-sensitive dye is an oxygen sensitive phosphor, and particularly one of the above-mentioned oxygen sensitive phosphors, then the oxygen sensitive phosphor phosphoresces.

Without wishing to be bound by theory, the as the one or microorganisms that are inoculated in the growth compartment respire and reproduce, they can consume the oxygen within the growth compartment. Because the culture device is in the closed configuration, the consumed oxygen cannot be replaced by oxygen from the exterior of the growth compartment and thus the partial pressure of oxygen within the growth compartment decreases. When the partial pressure decreases sufficiently then the oxygen sensitive-dye undergoes a color-change, which in the case of an oxygen-sensitive luminophore, particularly an oxygen sensitive phosphor, includes exhibiting detectable luminescence, such as phosphorescence.

The color change, and particularly luminescence, such as phosphorescence, can be in any detectable wavelength and does not need to be in the visible spectrum. A detectable wavelength is a wavelength that can be detected by a detector. A variety of light detectors are known to the artisan, and a suitable detector, for example a charge coupled device (CCD), photodiode, or even a human eye, may be selected depending on the wavelength of luminescence. When the color change is a change in absorption, it can be measured by absorption spectroscopy such as UV/VIS absorption, IR absorption, or the like.

It is also possible to enumerate the microorganisms. This can be accomplished with any of the culture devices or methods described above, and is simplest to do when the oxygen sensitive dye is an oxygen-sensitive luminophore that is homogenously distributed in the culture medium, in an adhesive, or on an adhesive. Enumerating can be performed, for example, by using a detector, such as a CCD camera, to record a picture of the entire growth compartment of the culture device that measures the intensity, location, or both intensity and location of the luminescence. The number of colony forming units can then be counted from the picture, for example, by assigning areas having an intensity that is higher than a threshold intensity to represent a colony. The threshold intensity will depend on the particular culture device and microorganism, but will be an intensity that differentiates between the presence of microorganism and noise. The concentration of the oxygen in any area of the growth compartment can also be determined indirectly, for example, by determining the oxygen concentration at particular locations in the growth compartment. The oxygen concentration at any location in the growth compartment, which can be related to the quantity of microorganisms in that location, can be calculated using the Stern-Volmer relationship.

Notably, these methods are preferably conducted without placing the culture device, or more particularly the growth compartment of the culture device, in a reduced oxygen atmosphere, such as a glove box. Further, these methods are preferably conducted without activating an oxygen scavenging system within the culture device, or more particularly within the growth compartment of the culture device.

1. A culture device comprising
a growth compartment surrounded by one or more oxygen impermeable barriers, at least one of the oxygen impermeable barriers being configurable between an open configuration where the growth compartment is exposed to an environment outside of the growth compartment and a closed configuration wherein the growth compartment is sealed from exchanging oxygen with the environment outside of the growth compartment;
a culture medium capable of supporting replication of at least one microorganism disposed within the growth compartment; and
an oxygen-sensitive dye disposed within the growth compartment.

2. The culture device of claim 1, wherein the oxygen sensitive dye comprises an oxygen sensitive luminophore.

3. The culture device of claim 1, wherein the at least one of the oxygen impermeable barriers is a coverslip that is configurable between a first position where the coverslip is on the growth compartment and a second configuration wherein the coverslip is at least partially detached from the growth compartment.

4. The culture device of claim 1 wherein the culture medium comprises agar or a water-gelling thin film.

5. The culture device of claim 1 wherein the oxygen-sensitive luminophore is an oxygen-sensitive phosphor.

6. The culture device of claim 1, wherein the oxygen-sensitive phosphor comprises at least one of a porphyrin, a pi-conjugated molecule, or a pi-conjugated polymer.

7. The culture device of claim 6, wherein the oxygen-sensitive phosphor comprises a porphyrin.

8. The culture device of claim 1, wherein the oxygen-sensitive dye comprises a pi-conjugated ligand.

9. The culture device of claim 2, further comprising a metal conjugated to the oxygen sensitive luminophore, wherein the metal is optionally a transition metal or lanthanide.

10. The culture device of claim 8, wherein the pi-conjugated molecule comprises a porphyrin.

11. The culture device of claim 2, wherein the oxygen sensitive luminophore is Pd-meso-tetra-(4-carboxypenyl)porphyrin dendrimer.

12. The culture device of claim 1, wherein the oxygen-sensitive dye comprises leuco-form indigo dye, leuco-form thioindigo dye, one or more complexes of bis(histadino) cobolt, or meso-tetra(α-α-α-α-o-pivalminophenyl) porphyrinatocobolt.

13. The culture device of claim 1, wherein no oxygen scavenging system is present within the growth compartment.

14. The culture device of claim 2, wherein the oxygen sensitive luminophore is present in the culture medium.

15. The culture device of claim 1, further comprising one or more adhesive matrixes within the growth compartment and wherein the oxygen sensitive luminophore is dispersed within at least one of the one or more adhesive matrixes.

16. A method of detecting a microorganism comprising;
inoculating a culture device of claim 1 with a sample comprising a microorganisms while the culture device is in the open configuration;
converting the culture device to the closed configuration;
incubating the culture device for a sufficient time and at a sufficient temperature such that the oxygen-sensitive phosphor phosphoresces; and
detecting a change of absorption or emission of the oxygen-sensitive dye.

17. The method of claim 16, wherein the change of absorption of emission is a change in emission, of an oxygen sensitive luminophore.

18-19. (canceled)

20. The method of claim 16, wherein the method further comprises enumerating the microorganism.

21. The method of claim 20 wherein enumerating the microorganism comprises determining a magnitude of the luminescence.

22. The method of claim 16, wherein the method does not include a step of placing the culture device within a reduced oxygen atmosphere.

23. The method of claim 16, wherein the method does not include a step of activating an oxygen scavenging reagent within the growth compartment.
